(19) United States
(12) Patent Application Publication
(10) Pub. No.: US 2024/0218786 A1
(43) Pub. Date: Jul. 4, 2024

(54) APPARATUS AND METHOD FOR MONITORING CHARACTERISTICS OF FLUIDS

(71) Applicant: GeoKimika Oil & Gas, LLC, Spring, TX (US)

(72) Inventors: Tanhee Galindo, Spring, TX (US); Kristin Hatch, Midland, TX (US)

(21) Appl. No.: 18/400,080

(22) Filed: Dec. 29, 2023

Related U.S. Application Data
(60) Provisional application No. 63/514,871, filed on Jul. 21, 2023.
(60) Provisional application No. 63/478,393, filed on Jan. 4, 2023.

(51) Int. Cl. E21B 47/10; E21B 43/26; E21B 47/017; E21B 47/12; E21B 47/26; G01N 33/28

(52) U.S. Cl. CPC E21B 47/10; E21B 43/26; E21B 47/017; E21B 47/12; E21B 47/26; G01N 33/2823

(57) ABSTRACT

Apparatus, systems, and methods for real-time monitoring of fluid characteristics. The apparatus includes manifolds comprising a conduit configured for fluid flow therethrough and one or more sensor ports configured to equip corresponding sensors through an external surface of the conduit. The systems and methods utilize the manifolds, as well as associated sensors and a monitoring device, which may communicate fluid characteristic data to external devices operated by both on-site and off-site users. The apparatus, systems, and methods are particularly useful for monitoring characteristics of fluids used in downhole operations, for example, before introduction into a well.

### RELATED APPLICATIONS

This application claims the benefit of U.S. Provisional Patent Application No. 63/478,393 filed Jan. 4, 2023, and U.S. Provisional Patent Application No. 63/514,871, filed Jul. 21, 2023, both of which are incorporated by reference herein in their entireties.

### BACKGROUND OF THE INVENTION

### Field of the Invention

The present invention relates to systems, methods, and related apparatus for real-time detection and monitoring characteristics of fluids. Embodiments of the present invention are particularly useful for on-site monitoring of fluid properties in downhole operations.

### Description of Related Art

Downhole operations utilize various fluid streams, such as hydraulic fracturing fluid, drilling fluid (drilling mud), and other fluid streams, each of which must have particular characteristics for the specified process. For example, hydraulic fracturing fluid and drilling mud will generally be formulated to comprise predominantly water having a specific concentration of acids, solids (e.g., sand), and/or other additives. Additionally, wastewater will generally include undesirable components that must be removed, neutralized, or otherwise treated before recycling the wastewater for use in other processes or releasing to the environment. Operators must have accurate information about the compositions and other characteristics of such fluid streams to ensure efficient and safe operations.

Testing of hydraulic fracturing fluid, drilling mud, and other related fluids such as source water, wastewater, etc., is typically performed by a testing service or laboratory located in a different city or state from the drill site. Fluid samples must therefore be collected on site and shipped to the testing facility, which can cause significant delays in testing and receiving results. Thus, sampling and testing of these fluids is typically performed days or weeks in advance of its use or discharge. This delay limits the ability to perform quality audits or troubleshoot chemical performance issues in the field.

### SUMMARY OF THE INVENTION

The present invention is broadly concerned with fluid quality monitoring apparatus, devices, systems, and methods that overcome one or more of the problems described above. The devices, systems, and methods are operable to provide real-time data, particularly compositional properties of the fluid, to on-site and/or off-site operators and may be particularly useful in extreme environments, such as high flow rates, high salinity, high corrosive, and high solids content fluids (e.g., hydraulic fracturing fluid, drilling mud, etc.).

In one embodiment, there is provided a sensor manifold. The manifold comprises a conduit comprising an inlet and an outlet and a passage extending therebetween, and configured to allow a fluid stream to flow within the passage, and one or more sensor ports formed in the conduit between the inlet and the outlet. Each of the one or more sensor ports communicates with the passage and comprises an outwardly extending projection configured to receive a sensor and to place the sensor in contact with the fluid stream flowing through the passage.

In another embodiment, there is provided a sensor manifold. The manifold comprises a conduit comprising an inlet and an outlet and a passage extending therebetween and configured to allow a fluid stream to flow through the passage, one or more baffles positioned within the conduit between the inlet and the outlet, and operable to impede and/or direct the fluid stream flowing through the conduit, and one or more sensor ports formed in the conduit between the inlet and the outlet. Each of the one or more sensor ports communicates with the passage. At least one of the one or more sensor ports is positioned downstream of at least one of the one or more baffles.

In another embodiment, there is provided a device for monitoring one or more characteristics of a fluid stream detected by one or more sensors. The device comprises a control module comprising one or more processing elements, one or more communication modules, and a case housing the control module and the one or more communication modules. The control module is configured to receive a signal generated by the one or more sensors which is representative of a condition of a fluid stream flowing through a manifold equipped with the one or more sensors, and to convert the signal to transmittable fluid characteristic data. The one or more communication modules are configured to receive the fluid characteristic data from the control module and to communicate the fluid characteristic data to one or more external devices. The case is configured to protect the control module and the one or more communication modules from an external environment.

In another embodiment, there is provided a system for monitoring one or more characteristics of a fluid. The system comprises one or more manifolds, one or more sensors equipped to the one or more sensor ports and operable to detect one or more characteristics of a fluid stream flowing through the conduit, and a monitoring device configured to receive a signal representative of the one or more characteristics from the one or more sensors. The one or more manifolds each comprise a conduit comprising an inlet and an outlet and a passage extending therebetween, and configured to allow a fluid stream to flow within the passage, and one or more sensor ports formed in the conduit between the inlet and the outlet, each of the one or more sensor ports communicating with the passage.

In another embodiment, there is provided a method of monitoring one or more characteristics of a fluid stream. The method comprises flowing the fluid stream through a manifold, detecting, via one or more sensors equipped to the one or more sensor ports of the manifold, a condition of the fluid stream flowing through the manifold, each of the one or more sensors generating a signal representative of the condition, and transmitting the signal representative of the condition to a monitoring device. The manifold comprises a conduit comprising an inlet and an outlet and a passage extending therebetween through which the fluid stream to flows, and one or more sensor ports formed in the conduit between the inlet and the outlet, each of the one or more sensor ports communicating with the passage.

In another embodiment, there is provided a computer implemented method for monitoring one or more characteristics of a fluid stream flowing through a manifold. The method comprises:
providing a monitoring device comprising a control module comprising one or more processing elements, one or more communication modules, and a case housing the control module and the one or more communication modules;
detecting, via one or more sensors equipped to the manifold, a condition of the fluid stream flowing through the manifold;
generating, via the one or more sensors, a signal representative of the condition;
receiving, via the control module, the signal representative of the condition;
converting, via the control module, the signal representative of the condition to transmittable fluid characteristic data;
communicating, via the one or more communication modules, the transmittable fluid characteristic data to one or more external devices.

### BRIEF DESCRIPTION OF THE DRAWINGS

FIG. 1 is a perspective view of a hydraulic fracturing fluid discharge system including a fluid monitoring system in accordance with embodiments of the present invention;

FIG. 2 is a schematic of a hydraulic fracturing fluid discharge system to a downhole well apparatus including a fluid monitoring system in accordance with embodiments of the present invention;

FIG. 3 is a schematic showing the internal components of a control unit in accordance with embodiments of the present invention;

FIG. 4 is a perspective view of a manifold in accordance with embodiments of the present invention;

FIG. 5 is a perspective cutaway view of the manifold of FIG. 4;

FIG. 6 is a side cross-sectional view of the manifold of FIG. 4;

FIG. 7 is a perspective cutaway view of a manifold in accordance with embodiments of the present invention;

FIG. 8 is a side cross-sectional view of the manifold of FIG. 7;

FIG. 9 is a side cross-sectional view of an alternative baffle arrangement with the manifold of FIG. 7; and

FIG. 10 is a schematic of an exemplary process for collecting, monitoring, and reporting fluid characteristic data in accordance with embodiments of the present invention.

### DETAILED DESCRIPTION

The present invention is concerned with apparatus and methods for monitoring characteristics of a fluid stream, for example a water or other liquid stream, as well as systems utilizing the apparatus and methods. The systems generally comprise one or more sensor manifolds for directing a fluid stream, one or more sensors equipped to the one or more manifolds operable to detect one or more characteristics of the fluid stream, and a device for monitoring the one or more characteristics of the fluid stream and configured to receive data from the one or more sensors. In certain embodiments, the fluid stream may comprise a liquid or multi-phase fluid stream. In certain embodiments, the fluid stream may comprise an aqueous (water-based) fluid having components dissolved, suspended, or otherwise present therein other than water. For example, the fluid stream may comprise an aqueous (water-based) fluid comprising water as the majority component (i.e., at least 50%, at least 60%, at least 70%, at least 80%, at least 90%, at least 95%, at least 97%, at least 98%, or at least 99% by weight), which may further comprise sand, alcohols (e.g., methanol, ethylene glycol), thickening agents, acids (e.g., hydrochloric acid, acetic acid), salts (e.g., sodium chloride), friction reducers (e.g., polyacrylamide), anti-corrosion chemicals, anti-microbials, foaming agents, scale inhibitors, and/or pH buffers (e.g., KOH, etc.). The apparatus, methods, and systems described herein are therefore particularly suitable for real-time testing and monitoring of characteristics of the fluid stream, such as acidity (pH), oxidation-reduction potential (ORP), temperature, conductivity, total dissolved solids, scaling, corrosion, turbidity, dissolved oxygen, dissolved carbon dioxide, analyte concentrations, pressure, flow rate, specific gravity, oxidizer concentration (e.g., chlorine, chlorine dioxide, peracetic acid, etc.), and/or others. Sensor readings for such characteristics can provide on-site and off-site operators with valuable information regarding fluid quality, fluid conditions, sediment (e.g., sand) levels, and/or chemical levels of various fluids related to hydraulic fracturing operations. However, it should be understood that the apparatus, methods, and systems described herein may be utilized in other applications where real-time collection and analysis of fluid characteristics are needed or desired.

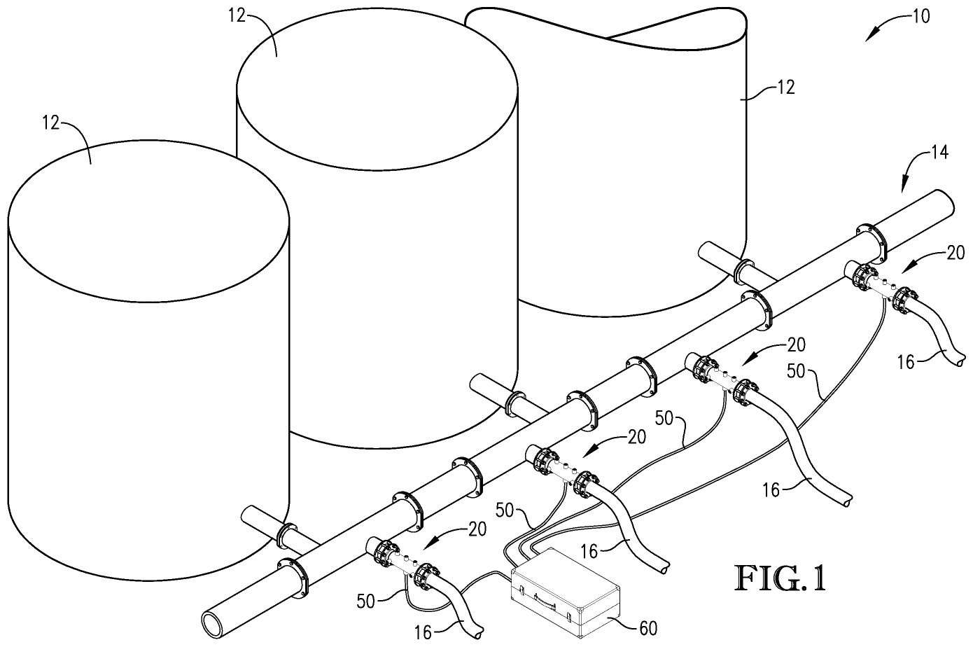

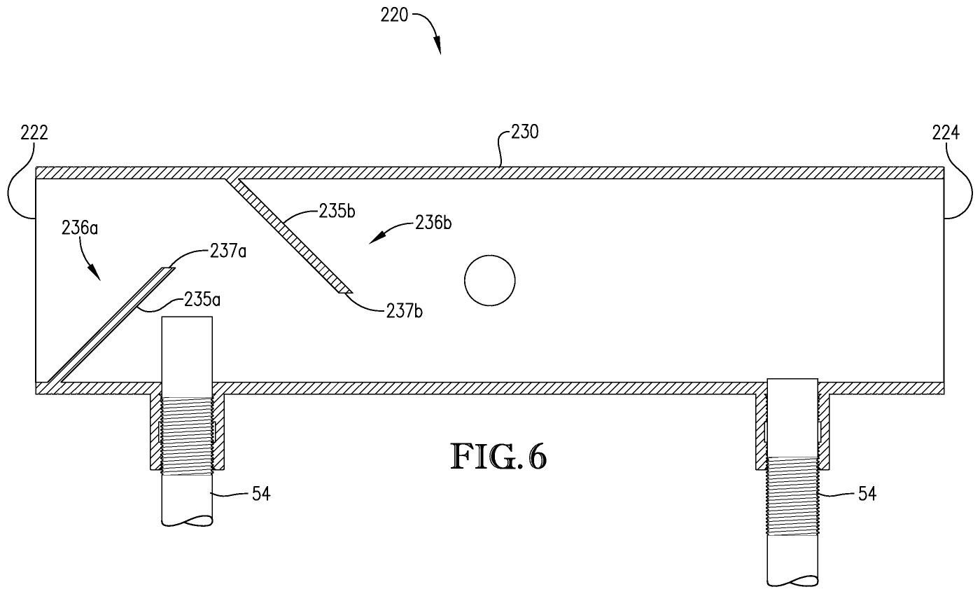

An exemplary system 10 is shown in FIG. 1. As shown in FIG. 1, system 10 comprises manifolds 20, equipped with sensors (installed in the manifolds 20—see FIG. 6) connected via wires 50 to monitoring device 60. Although FIG. 1 depicts sensors connected to monitoring device 60 via wires 50, wireless sensors may also be used to communicate signals to the monitoring device 60. Each manifold 20 is installed between one or more holding tanks 12, which can feed a fluid (e.g., hydraulic fracturing fluid) to a shared discharge conduit 14, and corresponding discharge lines 16. Thus, the manifolds 20 may be advantageously installed in a position downstream of tanks 12 to allow the installed sensors to take and transmit readings of the fluid being discharged via lines 16. Although FIG. 1 depicts a holding tank 12 for holding the fluid, it should be understood that other fluid reservoirs may also hold the fluid, such as ponds, lakes, or aquifers.

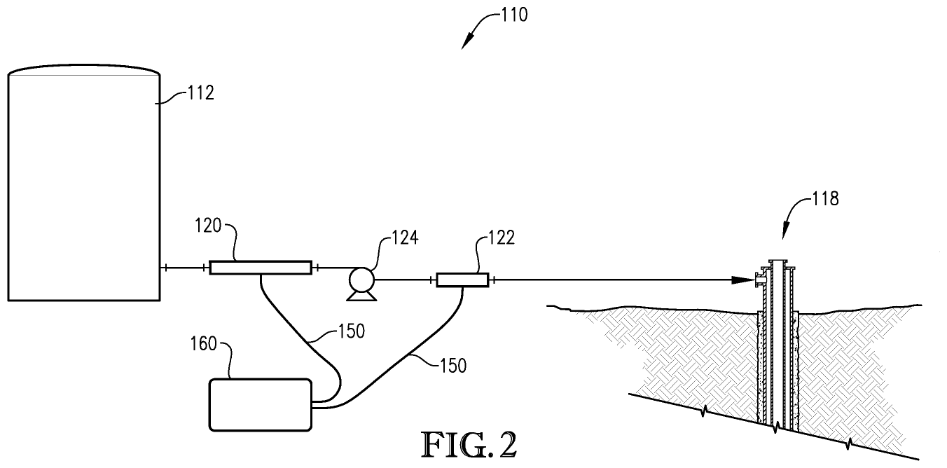

FIG. 2 depicts an exemplary application for the apparatus, methods, and systems described herein. In particular, as shown in FIG. 2, a system 110 is provided for monitoring the characteristics of a fluid stream being discharged from fluid holding tank 112 into a downhole well structure 118. The fluid stream may comprise, for example, a hydraulic fracturing fluid, a drilling mud, or other fluid stream. In certain embodiments, the methods may thus generally comprise include discharging a fluid stream from a holding tank 112, flowing the fluid stream through one or more manifolds 20, monitoring one or more characteristics of the fluid stream by transmitting a signal generated by one or more sensors equipped to the one or more manifolds 20 to a monitoring device 60, and optionally, introduce the fluid stream into a downhole well structure 118. In certain embodiments, one or more intervention steps may be utilize to adjust the characteristics of the fluid stream, for example, if the monitoring steps detect a condition outside of an acceptable or desired range.

As shown in FIG. 2, system 110 comprises an upstream manifold 120 and a downstream manifold 122, each equipped with a least one sensor installed therein. One or more pumps 124 and/or other equipment may be installed between upstream manifold 120 and downstream manifold 122. For example, although FIG. 2 depicts a single pump 124 between upstream manifold 120 and downstream manifold 122, it should be understood that pump 124 may comprise multiple pumps in parallel and/or series, which may also be, additionally or alternatively, located upstream of manifold 120 and/or downstream of manifold 122. Other equipment may include, but is not limited to, filters, valves (e.g., flow control, purge, etc.), and/or chemical injection devices. In certain such embodiments, system 110 can therefore provide sensor data related to the fluid characteristics both upstream and downstream of the pump 124 and/or other equipment. The sensors installed in the manifolds 120, 122 can be connected via wires 150 to monitoring device 160, although wireless sensors may also be used.

### Manifolds

Manifolds according to embodiments of the present invention generally comprise a conduit comprising an inlet and an outlet defining a passage extending therebetween and configured to allow fluid flow through the passage from the inlet to the outlet. One or more sensor ports may be formed in the conduit of the manifold between the inlet and the outlet. Each of the sensor ports may be configured to equip a sensor as described herein.

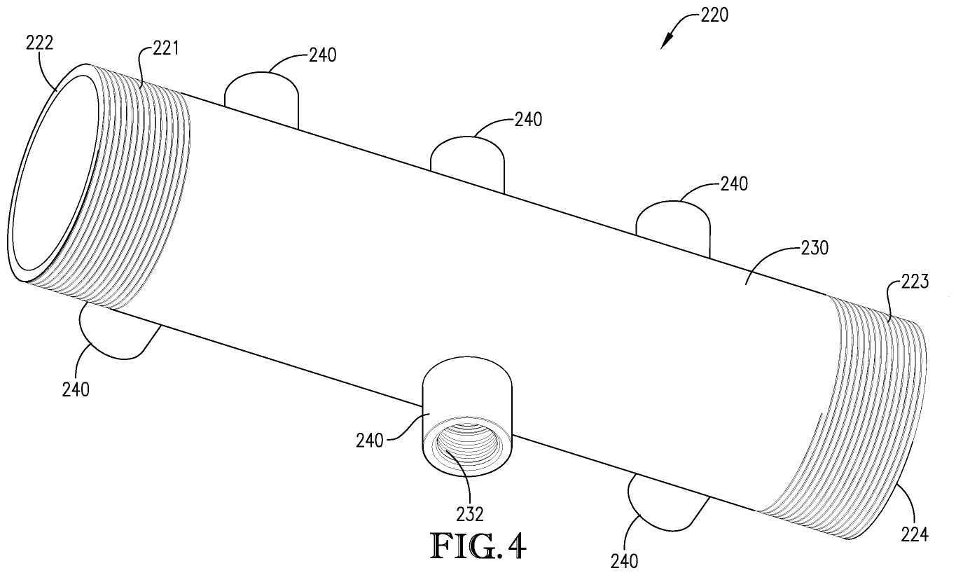

An exemplary manifold 220 is shown in FIG. 4. As shown, manifold 220 comprises conduit 230 having an inlet 222 and an outlet 224. In certain embodiments, conduit 230 may be a substantially tubular construction (e.g., a substantially symmetrical circular cylinder). Inlet 222 and outlet 224 may each comprise threaded portions 221, 223, for example on an external surface of the conduit 230, which may be used to threadedly secure the manifold to piping associated with the system, such as shared discharge conduit 14 or discharge lines 16.

Manifold 220 further comprises one or more sensor ports 240 communicating with the passage of conduit 230. In certain embodiments, the sensor ports 240 may be in the form of an outwardly extending projection (as shown) configured to receive a sensor, for example installed in a corresponding sensor port 240 through the external surface of the conduit 230, and to place the sensor in contact with the fluid flowing through the passage of conduit 230 to detect one or more characteristics of the fluid flowing therethrough. In certain embodiments, the projection may comprise an internal threaded portion 232, which may be used to threadedly secure a corresponding sensor within the sensor port 240 such that the measurement surface of the sensor is at the desired depth within the conduit, as described below. As shown, several sensor ports 240 may be included and located at various positions along the conduit 230, which can allow for the installation of sensors at advantageous positions to obtain the best readings for selected sensors.

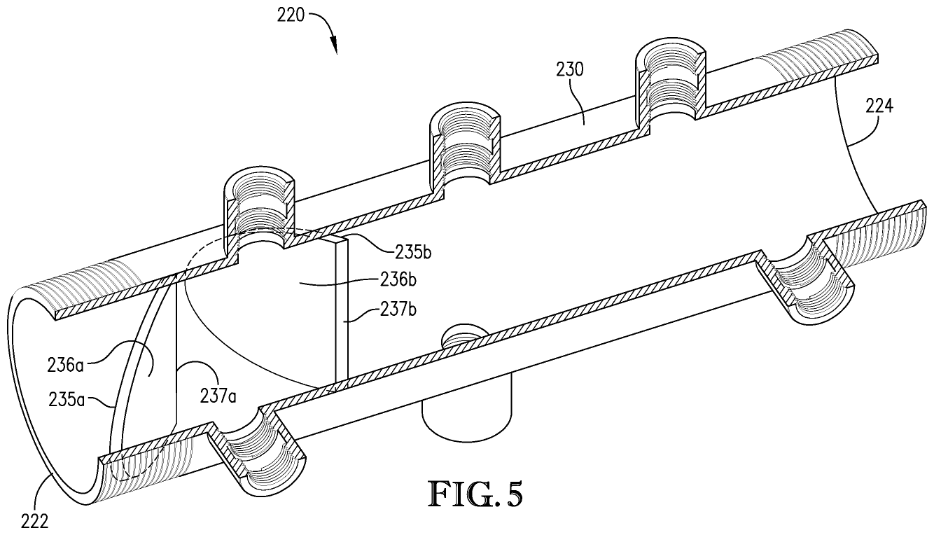

FIG. 5 shows the internal components of manifold 220. As shown, one or more baffles 236a, 236b may be positioned within the passage of conduit 230 between inlet 222 and outlet 224. Although the embodiments shown in FIG. 5 through FIG. 8 comprise two baffles, it should be understood that, in certain embodiments, only one baffle may be present or more than two baffles may be present. In use, the one or more baffles are configured to impede and/or direct the fluid flow in a particular direction through conduit 230. In certain embodiments, each of the baffles 236a, 236b may comprise a planar member having an arcuate edge 235a, 235b abutting the inner surface of conduit 230 and a free edge 237a, 237b disposed within the passage. Thus, each baffle 236a, 236b may be positioned so as to partially block the fluid flow through the passage and to permit fluid flow through the gap between the free edge 237a, 237b and the inner surface of conduit 230. In certain embodiments, the baffles 236a, 236b may have a substantially symmetrical geometry across a central axis running from the arcuate edge to the free edge.

In certain embodiments, when viewed down the passage of conduit 230 (i.e., looking through inlet 222), the arced edge 235a, 235b of the partial disc baffles 236a, 236b may appear to have a substantially circular arc. In certain embodiments, the arcuate edge 235a, 235b may have an arc angle of at least about 180° (e.g., a semi-circle). In certain embodiments, the free edge 237a, 237b may extend past a central longitudinal axis of the passage and thus the arcuate edge 235a, 235b may have an arc angle of greater than 180°, at least about 200°, at least about 210°, at least about 220°, at least about 230°, at least about 240°, at least about 250°, at least about 260°, or at least about 270°. In certain embodiments, the arcuate edge 235a, 235b may have an arc angle of about 150° to about 300°, about 180° to about 270°, about 200° to about 250°, or about 210° to about 230°. Although when viewed down the passage of conduit 230 (i.e., looking through inlet 222), the arced edge 235a, 235b of the baffle 236a, 236b may have a substantially circular arc, it should be understood that the one or more baffles may have an irregular circular shape (i.e., elliptical shape) to fit within the conduit, for example, when the baffles are inclined, as described below.

In certain embodiments, manifold 220 may comprise at least a first baffle 236a and a second baffle 236b. In certain such embodiments, the second baffle 236b may be positioned downstream of the first baffle 236a (i.e., second baffle 236b is closer to outlet 224 than first baffle 236a is to outlet 224) within conduit 230, such as shown in the embodiments of FIG. 5 through FIG. 8. As best shown in FIG. 6, in certain embodiments, the one or more baffles 236a, 236b may comprise at least one (or two, or more) inclined baffle(s) obliquely positioned relative to a central longitudinal axis of the passage. As used herein, the term “inclined baffle” refers to a planar member within the conduit having a plane obliquely positioned at a non-normal angle (i.e., an angle of less than or greater than 90°) relative to a central longitudinal axis of the passage conduit 230.

In certain embodiments, at least one (or two, or more) of the inclined baffle(s) is angled toward the outlet 224 (i.e., with the free edge 237a, 237b pointed toward the outlet 224). In certain embodiments, at least one (or two, or more) of the inclined baffle(s) is angled toward the inlet 222 (i.e., with the free edge 237a, 237b pointed toward the inlet 222).

In certain embodiments, such as shown in FIG. 6, at least a first baffle 236a and a second baffle 236b are each positioned in the upstream half of conduit 230 closer to inlet 222 than to outlet 224. In certain same or other embodiments, at least a first baffle 236a and a second baffle 236b are each angled toward the outlet 224 of conduit 230. In certain same or other embodiments, the first baffle 236a is positioned within the conduit 230 such that free edge 237a extends past a central longitudinal axis of the passage, and the second baffle 236b is positioned within the conduit 230 opposite the first baffle 236a such that free edge 237b extends past the same central longitudinal axis of the passage on an opposite side from the first free edge 237a. For example, when viewed from the side view angle of FIG. 6, first baffle 236a appears to extend from the bottom-most inner surface of conduit 230 and second baffle 236b appears to extend from the upper-most inner surface of conduit 230. In such embodiments, the free edge 237a of baffle 236a and the free edge 237b of baffle 236b are offset but substantially parallel within the passage.

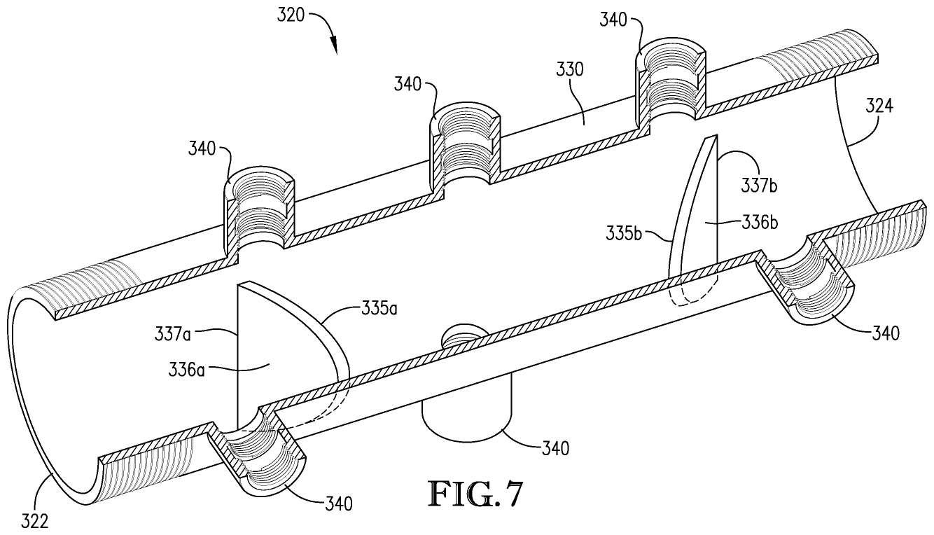

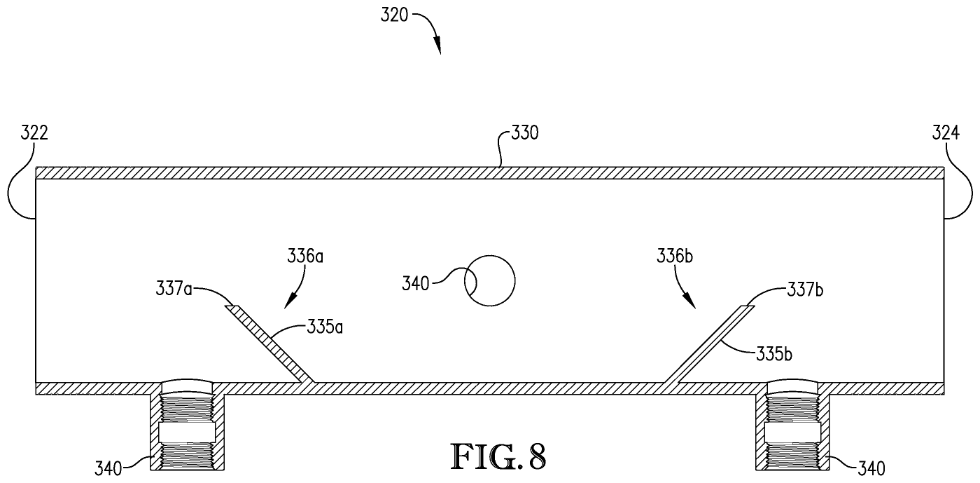

An alternative manifold 320 is shown in the embodiment of FIG. 7 and FIG. 8. As shown, in certain embodiments, at least a first baffle 336a is positioned in the upstream half of conduit 330 closer to inlet 322 than to outlet 324, and at least a second baffle 336b is positioned in the downstream half of conduit 330 closer to outlet 324 than to inlet 322. In certain such embodiments, the first baffle 336a may be spaced from inlet 322 at distance substantially equal to the distance from which second baffle 336b is spaced from outlet 324. In certain same or other embodiments, at least a first baffle 336a is angled toward the inlet 322 of conduit 330, while and at least a second baffle 336b is angled toward the outlet 324 of conduit 330. In certain same or other embodiments, at least a first baffle 336a and a second baffle 336b are positioned within conduit 330 such that both free edge 337a and free edge 337b each extend in the same direction past a central longitudinal axis of the passage. For example, when viewed from the side view angle of FIG. 8, first baffle 336a and second baffle 336b appear to extend from the bottom-most inner surface of conduit 330. In such embodiments, the arcuate edge 335a of baffle 336a and the arcuate edge 337b of baffle 336b will appear substantially aligned and parallel within the passage.

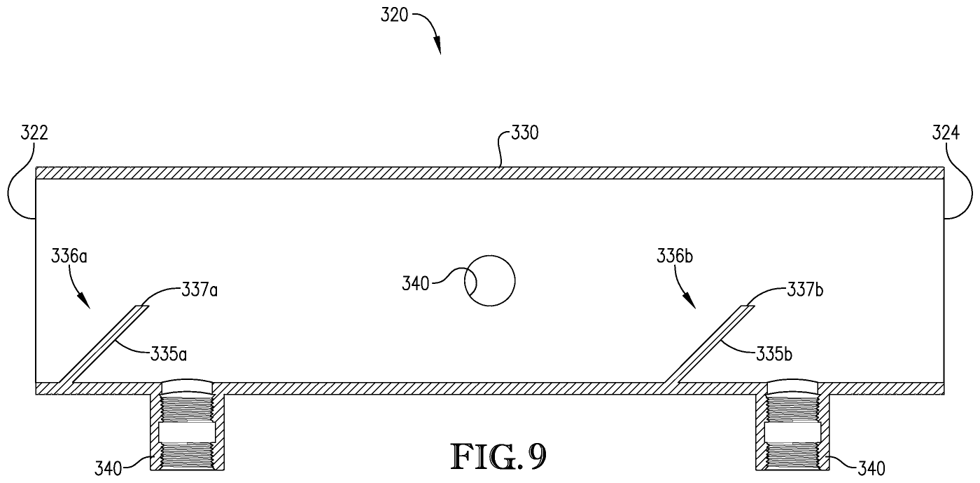

An alternative baffle arrangement in manifold 320 is shown in FIG. 9. As shown, in certain embodiments, the first baffle 336a is positioned upstream of all ports 340. In certain such embodiments, at least the first baffle 336a is angled toward the outlet 324 of conduit 330. In certain such embodiments, at least the second baffle 336b may also be angled toward outlet 324 of conduit 330.

Without being bound by any theory, it is believed that the use and positioning of baffles, as described herein, impedes and/or directs the fluid flowing through the conduits so as to allow the sensors installed therein to take more accurate readings of the characteristics of the fluid stream. For example, in certain embodiments, the baffles may be aligned upon installation to drive the fluid toward a particular sensor or sensors. The baffles may also protect a sensor or sensors from damage and/or erosion by deflecting solids entrained in the flowing fluid. Thus, the manifold may be oriented upon installation to ensure the fluid flows over certain baffles and/or sensors. In certain embodiments, at least one of the one or more sensor ports may be positioned downstream of at least one of the one or more baffles such that the fluid flow impeded by the one or more baffles facilitates a more accurate sensor reading. In certain embodiments, at least one of the one or more sensor ports is positioned downstream of at least a first baffle and a second baffle (see FIG. 6). In certain embodiments, at least one of the one or more sensor ports is positioned downstream of at least a first baffle and upstream of at least a second baffle (see FIG. 8).

### Sensors

Sensors used in accordance with embodiments of the present invention are generally configured to generate a signal representative of a condition of the fluid flowing through manifolds 20 and contacting the installed sensor sensitive to the particular condition. As described below, the sensors are in communication (wired or wireless) with monitoring device 60. As shown in FIG. 1, the sensors installed in manifolds 20 may be in communication with monitoring device 60 via wires 50 that transmit the signal generated by the sensors to one or more processing elements, one or more memory elements, and/or one or more communication modules within monitoring device 60.

In certain embodiments, the sensors may be installed, for example, by securing the sensors within the projections of ports 240 of manifold 220. The sensors 54 may be individually installed such that the measuring surface of each sensor is at the desired depth within the conduit 230. For example, as shown in FIG. 6, the sensors 54 may be installed at differing depths, depending on the type of sensor and position along the conduit. In certain embodiments, one or more sensors 54 may be installed substantially flush with the inner surface of the conduit. Sensor installed substantially flush with the inner surface of the conduit may be advantageously protected from damage by solids flowing through the conduit. In certain embodiments, one or more sensors 54 may be installed such that the measuring surface is at a depth of at least ½ an inch, at least 1 inch, at least 1½ inches, or at least 2 inches into the conduit, which may provide for more accurate readings of certain fluid conditions.

Exemplary sensors are configured to detect fluid conditions such as acidity (pH), oxidation-reduction potential (ORP), temperature, conductivity, total dissolved solids, scaling, corrosion, turbidity, dissolved oxygen, dissolved carbon dioxide, analyte concentrations, pressure, flow rate, as well as others. For example, the sensors may be EZO circuit sensors, such as those manufactured by Atlas Scientific, although other types of sensors may also be used, including but not limited to, analog sensors with 4-20 mA output or MODBUS 485 protocol. In certain embodiments, the sensors may comprise a titanium casing or other material that can withstand extreme environmental conditions, such as those conditions within hydraulic fracturing fluid discharge lines. Such environmental conditions can include high flow rates (e.g., >4000 gal/min), high salinity (e.g., about 100,000 to about 350,000 mg/L TDS), high corrosive (e.g., pH<about 5 or pH>about 9.5), and high solids (e.g., sand, debris, and other fracturing solids). In addition, the sensors can be configured to measure certain characteristics of the fluid, provide that data to the monitoring device which then uses the data to calculate the aforementioned fluid conditions. Furthermore, in certain embodiments sensor information can be combined with information calculated by the monitoring device to generate hybrid values for certain fluid conditions. For example, a sensor can be configured to take a direct measurement of the fluid corrosion value. However, data can also be collected by other sensors and then processed by the monitoring device to arrive at a calculated corrosion value. The sensed value can then be correlated with the calculated value to arrive at a hybrid corrosion value, which can be a more accurate reflection of the true corrosion value than either the sensed or calculated values alone.

### Monitoring Device

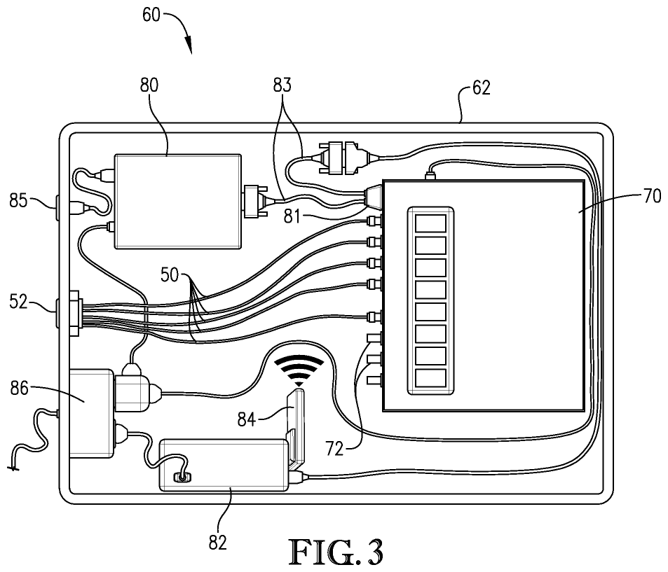

The monitoring device in accordance with embodiments of the present invention is generally in communication with the one or more sensors and is configured to receive the signal from the one or more sensors representative of a condition of the fluid flowing through manifolds 20. An exemplary embodiment of monitoring device 60, including the internal components, is best shown in FIG. 3. The internal components described herein may be housed in a case 62, which can be waterproof or otherwise protect the electronic components from the environment and/or include a handle to allow the user to easily transport monitoring device 60 to the desired location on-site or off-site. Thus, in certain embodiments, the monitoring device 60 is a portable device, which can be easily moved by a human operator. As shown, monitoring device 60 may comprise control module 70, a wired communication module 80, a wireless communication module 82 (which may be equipped with a wireless communication element 84), and a power source 86. When monitoring device 60 is installed, for example in system 10, wires 50 associated with the sensors may be fed through a sensor wire port 52 formed in case 62 such that the wires 50 can be installed to control module 70.

Control module 70 may generally comprise sensor input terminals 72, as well as one or more processing elements, one or more memory elements, and a user interface. The processing elements may be configured to receive a signal representative of a fluid condition generated by each sensor and to transmit the signal to one or more memory elements, a user interface, and/or one or more data communication devices. The one or more processing elements may include processors, microprocessors (single-core and multi-core), microcontrollers, DSPs, field-programmable gate arrays (FPGAs), analog and/or digital application-specific integrated circuits (ASICs), or the like, or combinations thereof. Each of the one or more processing elements may generally execute, process, or run instructions, code, code segments, software, firmware, programs, applications, apps, processes, services, daemons, or the like. The one or more processing elements may also include hardware components such as finite-state machines, sequential and combinational logic, and other electronic circuits that can perform the functions necessary for the operation of the current invention.

The one or more processing elements may be in communication with the other electronic components through serial or parallel links that include address busses, data busses, control lines, and the like. For example, the one or more processing elements of control module 70 may be in communication with one or more of the sensors (e.g., via wires 50, terminals 72, etc.), wired communication module 80, and/or wireless communication module 82. The one or more processing elements of the control module 70 may be configured to send and/or receive information to and/or from the above components. The one or more processing elements of the control module 70 may also be configured to send and/or receive commands to and/or from the above components.

The one or more memory elements may include data storage components, such as read-only memory (ROM), programmable ROM, erasable programmable ROM, random-access memory (RAM) such as static RAM (SRAM) or dynamic RAM (DRAM), cache memory, hard disks, floppy disks, optical disks, flash memory, thumb drives, universal serial bus (USB) drives, or the like, or combinations thereof. In some embodiments, the one or more memory elements may be embedded in, or packaged in the same package as, the one or more processing elements. The one or more memory elements may include, or may constitute, a “computer-readable medium”. The one or more memory elements may store the instructions, code, code segments, software, firmware, programs, applications, apps, services, daemons, or the like that are executed by the one or more processing elements.

The user interface generally allows the user to utilize inputs and outputs to interact with control module 70 and is in communication with the one or more processing elements. Inputs may include buttons, pushbuttons, knobs, jog dials, shuttle dials, directional pads, multidirectional buttons, switches, keypads, keyboards, mice, joysticks, microphones, or the like, or combinations thereof. The outputs of the present invention may include a display as well as any number of additional outputs, such as audio speakers, lights, dials, meters, printers, or the like, or combinations thereof, without departing from the scope of the present invention. In certain embodiments, the display comprises an electrophoretic portion configured to display an indication representative of an operating condition. The electrophoretic portion may comprise electronic ink, electronic paper, an electrophoretic display, electrophoretic ink, and/or an electrowetting display. The electrophoretic portion may be configured to display a single dot, a check mark, characters, codes, images, indicators, and/or the like, or combinations thereof.

Data from control module 70 may be transmitted to wired communication module 80 and/or wireless communication module 82, which may each comprise one or more processing elements and/or one or more memory elements, as described above. Each of the wired communication module 80 and/or wireless communication module 82 may be configured to receive data from control module 70, for example via serial splitter 81, which may be further connected to one or more serial cables 83. In certain embodiments, wired communication module 80 is configured to allow a user to plug an external device, such as a laptop, tablet, or other mobile device, into a port 85 providing wired communication of data from wired communication module 80 to the external device. Exemplary wired communication modules include NPort series devices manufactured by MOXA. Exemplary wireless communication modules include the Raspberry Pi single-board computing devices, which have been configured to facilitate wireless communication of data with one or more external devices.

Wireless communication module 82 may be equipped with a wireless communications element 84. The wireless communication element 84 may generally allow communication with systems or devices external to the monitoring device 60. The communication element 84 may include signal or data transmitting and receiving circuits, such as antennas, amplifiers, filters, mixers, oscillators, digital signal processors (DSPs), and the like. The communication element 84 may establish communication wirelessly by utilizing RF signals and/or data that comply with communication standards such as cellular 2G, 3G, 4G, 5G, or LTE, WiFi, WiMAX, Bluetooth®, BLE, or combinations thereof. The communication element 84 may also be in communication with the one or more processing elements and/or the one or more memory elements of control module 70, wired communication module 80, and/or wireless communication module 82.

In certain embodiments, the wireless communication module 82 may be in direct communication with one or more sensors (via additional wires 50) corresponding to one or more sensors. For example, wireless communication module 82 may comprise one or more input ports to which wires 50 may be installed. In such embodiments, the wireless communication module 82 may be configured to receive a second signal representative of a second condition of the fluid stream and/or a signal representative of a sensor condition (e.g., errors) generated by the one or more sensors. The wireless communication module 82 may then convert the signal to transmittable fluid characteristic data and/or sensor data, which may be wirelessly communicated (via the wireless communication element 84) to an external device. Such embodiments may permit, for example, collection of certain data by an off-site user that is not accessible to an on-site user via the wired communication module 80.

### Operation

Further embodiments of the present invention are directed to methods for monitoring one or more characteristics of a fluid stream, as well as related intervention steps, utilizing the apparatus and systems describe herein. An exemplary method of monitoring one or more characteristics of a fluid stream is described herein, but it will be understood that in some alternative implementations, the functions described may occur out of order or concurrently, certain functions may be omitted, and/or other functions may be included that are not described herein.

The method described below, for ease of reference, may be executed by exemplary apparatus and systems introduced with the embodiments illustrated in FIG. 1 through FIG. 8 and described herein. For example, the monitoring methods may utilize the sensors installed within one or more manifolds described herein, through which the fluid being monitored is flowing. Additionally or alternatively, the monitoring methods may utilize the monitoring device to process signals received from the sensors and to communicate associated data to a user via one or more communication modules.

In one or more embodiments, a method for monitoring one or more characteristics of a fluid stream is provided. The method generally comprises flowing a fluid stream through one or more manifolds as described herein. One or more sensors may be equipped to each of the one or more manifolds, for example installed into corresponding sensor ports in the manifolds. The method thus further comprises detecting one or more characteristics of the fluid flowing through the manifold conduit using the one or more equipped sensors. The monitoring device may receive a signal representative of a fluid characteristic from each sensor. In certain embodiments, the monitoring device may be configured to receive a signal from a sensor at designated time intervals, such as about 1 hour, about 1 minute, or about 1 second. In certain embodiments, the sensors may be calibrated or otherwise configured by a user from an external device in communication with the sensors via the monitoring device. In certain embodiments, the one or more manifolds are disposed between conduits of the primary or normal flow lines such that the sensors are configured to detect a condition of the fluid stream without diverting the fluid stream into a side stream for sampling (e.g., a separate sampling conduit or testing spool). This advantageously allows for real-time monitoring of the fluid without the need to recreate normal flow conditions for testing.

In certain embodiments, the fluid stream may comprise an aqueous fluid process stream, such as an aqueous fluid stream used in hydraulic fracturing operations. In certain embodiments, the fluid stream is a hydraulic fracturing fluid, which may be sourced, for example, from a holding tank and/or tanker truck. In certain embodiments, additives may be introduced to the fluid stream, for example upstream of one or more manifolds, as needed to produce a fluid having properties appropriate for the particular operation. As described herein, the amount of additives introduced to the fluid stream may be varied based on characteristic values identified by the sensors and communicated to the monitoring device and operator. The flow rate of the fluid stream through each manifold conduit may be controlled by one or more valves and/or pumps positioned upstream and/or downstream of the manifold. In certain embodiments, the flow rate through at least one (or all) of the one or more manifolds may be at least about 1000 gal/min, at least about 2000 gal/min, at least about 3000 gal/min, or at least about 4000 gal/min. In certain embodiments, the flow rate through at least one (or all) of the one or more manifolds may be about 100 to about 10,000 gal/min, about 1000 to about 8000 gal/min, or about 2000 to about 5000 gal/min.

One or more characteristics of the fluid flowing through the one or more manifolds may be detected by the sensors and can be communicated through the monitoring device to an on-site and/or off-site operator. For example, the steps of the signal detection and communication may be performed by the sensors and monitoring device described herein through the utilization of processors, transceivers, hardware, software, firmware, or combinations thereof. However, some of such actions may be distributed differently among such devices or other devices without departing from the spirit of the present invention. Control of the system may also be partially implemented with computer programs stored on one or more computer-readable medium(s). The computer-readable medium(s) may include one or more executable programs stored thereon, wherein the program(s) instruct one or more processing elements to perform all or certain of the steps outlined herein. The program(s) stored on the computer-readable medium(s) may instruct processing element(s) to perform additional, fewer, or alternative actions, including those discussed elsewhere herein. For example, in certain embodiments, the monitoring device (or one or more components therein) may have one or more programs installed thereon that specify how often to transmit signals from the sensors to the control module, that convert the signals into fluid characteristic data, and/or calculate a secondary data set using the fluid characteristic data from one or more of the sensors.

In certain embodiments, each of the one or more sensors equipped to the manifolds measures a condition on the fluid stream flowing through the manifolds and generates a signal that is representative of a fluid characteristic. For example, the generated signal may be representative of the acidity (pH), oxidation-reduction potential (ORP), temperature, conductivity, total dissolved solids, scaling, corrosion, turbidity, dissolved oxygen, dissolved carbon dioxide, analyte concentrations, pressure, flow rate, specific gravity, oxidizer concentration, or other characteristic of the fluid stream. Each of the generated signals from the one or more sensors may then be communicated to the monitoring device, via wired or wireless transmission. In particular, each generated signal may be communicated to a processing element of the control module within the monitoring device, which may convert the signal to data for storage in one or more memory elements and/or for transmitting to one or more communication modules. Additionally, in certain embodiments, the data converted by the control module may be further processed to calculate secondary data sets, for example, by using the converted data as variable in an equation for calculating another fluid characteristic value. For example, the data converted by the control module may be processed along with physical test data for the fluid to generate secondary data corresponding to the scaling potential for the fluid (e.g., Langelier saturation index, Stiff-Davis index, and chloride concentration). The physical test data can comprise data generated by laboratory testing of the same fluid coming into contact with the one or more sensors. For example, the physical test data can include, for example, values for the hardness and alkalinity of the fluid, and can then be processed along with sensor data pertaining to total dissolved solids, pH, temperature, and conductivity to determine the Langelier saturation index and/or Stiff-Davis index. The communication modules can then transmit the data (or secondary data) to wired (local) or wireless (remote) external devices, which can display an indication representative of the fluid characteristics detected by the sensors. It should be understood that the methods described herein may include additional, less, or alternate steps and/or device(s), including those discussed elsewhere herein.

In certain embodiments, the fluid characteristic data (or secondary data) converted by a processing element of the control module may be saved or stored on one or more memory elements of the control module and/or communication module. This data can then be recalled and communicated to an external device at a future time, thereby providing a history of real-time fluid characteristic data. By storing the fluid characteristic data (or secondary data) at least temporarily onto the one or more memory elements, gaps of missing data due to disconnection from the external devices can be avoided. Thus, data collection and reporting issues from accidental or intentional disconnection of the external devices from the communication module(s) are avoided.

Embodiments of the present invention are directed to monitoring systems and methods that allow for chemical control of the fluid stream composition. Therefore, in certain embodiments, the methods further comprise one or more intervention steps, which may be used to change one or more characteristics of the fluid stream in response to the fluid characteristic data generated during the monitoring steps described above. Exemplary intervention steps may include adjusting the flow rate of the fluid flowing through the manifolds and/or adjusting the concentration of one or more additives (such as those described herein) in the fluid stream. In certain such embodiments, the methods further comprise adjusting the amount of one or more additives introduced into the fluid stream. The adjusting step may comprise introducing a greater amount of a certain additive to the fluid stream, for example to increase the concentration of that additive in the fluid stream, or the adjusting step may comprise introducing a lesser amount of a certain additive to the fluid stream (or eliminating introduction of that additive altogether), for example to decrease the concentration of that additive in the fluid stream. In certain embodiments, the flow of the fluid stream through the manifold is reduced or ceased during one or more of the intervention steps.

Embodiments of the present invention are also directed to computer implemented methods of monitoring one or more characteristics of a fluid stream flowing through a manifold, which utilizes the apparatus, methods, and systems described herein. In certain embodiments, the computer implemented methods comprise providing a monitoring device (or other apparatus described herein) and are performed by components of the monitoring device, and optionally, in conjunction with one or more external devices (e.g., laptops, tablets, desktop computers). In certain embodiments, the computer implemented method generally comprises the following steps:
(1) detecting, via one or more sensors equipped to the manifold, a condition of a fluid stream flowing through the manifold;
(2) generating, via the one or more sensors, a signal representative of the condition;
(3) receiving, via the control module, the signal representative of the condition;
(4) converting, via the one or more processing elements of the control module, the signal representative of the condition to transmittable fluid characteristic data; and
(5) communicating, via the one or more communication modules, the transmittable fluid characteristic data to one or more external devices.
   Additionally or alternatively, the fluid characteristic data may be stored (and retrieved) on one or more memory elements of the control module and/or the one or more communication modules. In certain embodiments, the fluid characteristic data may be presented on the one or more external devices or provided as a printed report. Additionally or alternatively, the sensors may be calibrated by first collecting physical samples of the fluid stream and testing the fluid characteristics to provide calibration data points for the sensors and associated programs.

In certain embodiments, the fluid characteristic data may be communicated (and collected by a user) on-site and/or remote from the monitoring device. The data communicated on-site (locally) and off-site (remotely) may be the same or different data. For example, data communicated to remote external devices may be used to sensor and system monitoring and quality assurance, while data communicated locally can provide real-time fluid quality data for an on-site user. Audio and/or visual alarms may be configured to alert on-site and/or off-site users when errors (e.g., no data) or other notable events (e.g., fluid characteristics out of appropriate range) are detected by the sensors or monitoring device. Additionally or alternatively, the sensors and/or components of the monitoring device may be programmed or otherwise configured by an off-site user by communicating configuration programs and/or data to the sensor control device via the data processing device(s). When installing the sensors and control monitoring device components, such components may be labeled with a tracking code (e.g., QR code, barcode, etc.) so that programs operating on external devices can communicate with the tracked components.

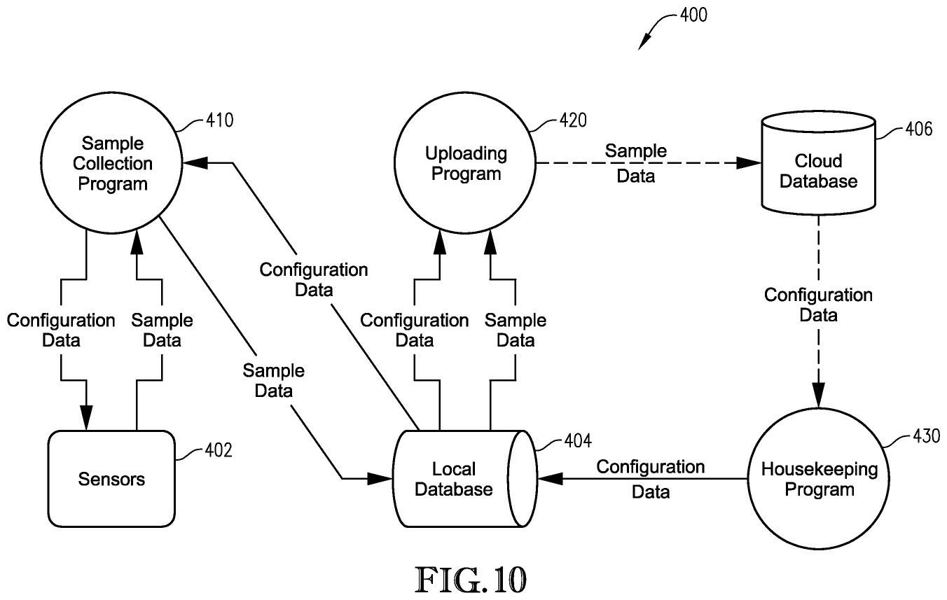

An exemplary process for collecting, monitoring, and reporting fluid characteristic data is schematically represented in FIG. 10 and described herein, including relevant hardware and associated programs for performing this process. It should be understood, however, that this exemplary process is not limiting on the overall scope of the invention, and other hardware components and programs not discussed herein may also be used in accordance with other embodiments of the present invention.

Referring to FIG. 10, the exemplary process 400 comprises three main programs configured to run concurrently on the data collection platform, including a sample collection program 410, an uploading program 420, and a housekeeping program 430. In certain embodiments, a system and service manager (e.g., Systemd) may be used to start and stop these programs, and aggregate their output into a common log. In certain embodiments, the programs may communicate through a database engine, such as a Sqlite database. While in certain embodiments, only one program can access the database at a given instant, the programs may be configured to repeatedly retry failed I/O operations, and thus all programs are tolerant of transient database or Internet errors. In certain embodiments, a program may exit after repeated fails, in which case the system and service manager will restart the failed program after a designated time period (e.g., 1 minute, 5 minutes, 10 minutes, 15 minutes, 30 minutes, or 1 hour). As described above, data may be stored on one or more memory elements of the monitoring device, and thus the programs may process the stored data upon restart without loss of data points to thereby provide a dataset with little or no gaps of missing data points.

The sample collection program 410 receives data in the form of processed signals from the one or more sensors 402 at prescribed intervals and saves the data to a sample table in the local database engine 404. The sample collection program receives as input a list of sensors to be read and their configuration from a sensors table in the local database engine 404. The sample collection program 410 also loads project metadata from the configuration table. This data is then saved with the sample reading into the sample table. The sample collection program itself does not require access to the Internet or any external network, as it runs continuously, even if the database is empty, damaged, or missing.

The sensors table and configuration table may be populated by an installation script, which may be refreshed at programmed time intervals (e.g., every 10 minutes) by the housekeeping program 430. These tables may be are queried every time the sample collection program 410 is booted, so it will receive the latest updates from the housekeeping program 430.

The sample collection program 410 may be configured to attempt one sample reading at a time, and subsequently checks the local database engine 404. If the sample collection program 410 has no immediate task, or an error occurs, it will pause (or sleep) for a designated time interval (e.g., 15 seconds) before retrying. The sample collection program 410 may be scheduled to operate in whole seconds (i.e., ˜1 second intervals). If the next scheduled sample time for a particular sensor is empty, the sample collection program 410 may move the sample time to immediately attempt a sample. If a sample deadline is missed by more than a designated time interval (e.g., ˜1 second), the sample collection program 410 may move the next sample attempt forward. This may happen, for example, when the sample collection program 410 attempted to read two sensors at the same time.

The uploading program 420 uploads data from collected samples to an external device, such as a cloud database 406. This can be accomplished, for example, by calling an Application Programming Interface (API) endpoint. In certain embodiments, the uploading program 420 can batch data from multiple samples together into a single API call. The uploading program 420 parameters are tunable so as to strike a balance between waiting to upload more samples at once, and not getting too far behind. For example, uploading program 420 may be configured to wait until there is data from about 2 to about 20 samples, about 5 to about 15 samples, or about 10 samples available to upload, unless a designated time period (e.g., 120 seconds) has elapsed since the last upload, in which it case it can upload the data for available samples.

The uploading program 420 verifies that each sample was written successfully using data returned from the API call. For each sample data point (or dataset) uploaded, the program writes the upload time back to the sample table. The housekeeping program 430 will periodically purge samples from the table that were uploaded older than a designated time period (e.g., more than 30 days ago).

Like the sample collection program 410, the uploading program 420 is tolerant of Internet or network outages and database errors. Sample data which fails to upload due to API errors, including API throttling, will be re-attempted and should succeed when the problem with the API resolves. Such errors may occur if sample data gets stuck, is unable to be properly formatted (due to missing or ill-formed sample data), or is unable to be saved to the cloud (due to a constraint violation on the back end). This should not happen on a normally functioning system, but if it occurs, it should not interfere with the flow of new samples, so long as the number of bad samples does not grow. If sample data errors accumulate, the offending samples may be manually deleted by the user to prevent uploads being attempted indefinitely. Data logs available to the user can provide detailed information on this when it occurs.

The housekeeping program 430 has several functions. First, it may purge old sample data (e.g., that was uploaded over 30 days prior) from the local database 404. This keeps the data in the local database 404 from growing indefinitely. Second, the housekeeping program 430 synchronizes the global configuration data and the sensor list with the cloud database 406. Finally, the housekeeping program 430 updates the program itself. This may be accomplished by pulling updates from a Git repository that was used to install the code. For example, the housekeeping program 430 may use the branch that was selected during installation (it does not change the current branch). Access to the Git repository may be obtained via a read-only access key.

In certain embodiments, updates to one or more of the programs may be downloaded to the housekeeping program 430, for example, from the cloud database 406. In certain embodiments, when the housekeeping program 430 detects that updates to the application code have been downloaded, the housekeeping program 430 can stop itself or other programs from running, update the installed code, and then restart the program(s) upon installation. The housekeeping program 430 advantageously avoids overwriting its own script, for example, by renaming the existing script first, then copying in the new one, and restarting the program that uses it.

The housekeeping program 430 may be configured to run in designated intervals (e.g., about every 10 minutes). While the background programs are stopped, or in the development environment, the housekeeping program 430 can be run on demand to update the local database 404. In certain embodiments, the background programs are not started in the development environment.

Although the apparatus, methods, and systems have been described herein having particular application to downhole operations (e.g., hydraulic fracturing, drilling, etc.), it should be understood that the apparatus, methods, and systems may have utility in various other applications. For example, these apparatus, methods, and systems may be useful in any applications where real-time monitoring of fluid characteristics is necessary or desirable.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if an apparatus is described as containing or excluding components A, B, and/or C, the apparatus can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

1. A system for monitoring one or more characteristics of a fluid, the system comprising:
one or more manifolds each comprising:
a conduit comprising an inlet and an outlet and a passage extending therebetween, and configured to allow a fluid stream to flow within the passage, and
one or more sensor ports formed in the conduit between the inlet and the outlet, each of the one or more sensor ports communicating with the passage;
one or more sensors equipped to the one or more sensor ports and operable to detect one or more characteristics of a fluid stream flowing through the conduit; and
a monitoring device configured to receive a signal representative of the one or more characteristics from the one or more sensors.

2. The system of claim 1, wherein the one or more sensor ports each comprise an outwardly extending projection configured to receive a sensor and to place the sensor in contact with the fluid stream flowing through the passage, wherein each of the one or more manifolds comprises one or more baffles positioned within the conduit between the inlet and the outlet, and operable to impede and/or direct the fluid stream flowing through the conduit, and wherein at least one of the one or more sensor ports is positioned downstream of at least one of the one or more baffles.

3. The system of claim 1, wherein the one or more sensors are configured to detect and/or provide data to the monitoring device that can be used to calculate acidity (pH), oxidation-reduction potential (ORP), temperature, conductivity, total dissolved solids, scaling, corrosion, turbidity, dissolved oxygen, dissolved carbon dioxide, analyte concentrations, pressure, and/or flow rate.

4. The system of claim 1, wherein the monitoring device comprises:
a control module comprising one or more processing elements configured to receive the signal generated by the one or more sensors and to convert the signal to transmittable fluid characteristic data;
one or more communication modules configured to receive the fluid characteristic data from the control module and to communicate the fluid characteristic data to one or more external devices; and
a case housing the control module and the one or more communication modules, and configured to protect the control module and the one or more communication modules from an external environment.

5. The system of claim 4, wherein the one or more communication modules comprises:
a wired communication module configured to provide wired communication of the fluid characteristic data to a first external device; and
a wireless communication module equipped with a wireless communication element and configured to provide wireless communication of the fluid characteristic data to a second external device, wherein the wireless communication module comprises one or more input ports to which wires corresponding to the one or more sensors may be installed and which are configured to receive a second signal generated by the one or more sensors which is representative of a second condition of the fluid stream.

6. The system of claim 4, wherein the control module comprises one or more memory elements configured to store the fluid characteristic data for a designated period of time.

7. A method of monitoring one or more characteristics of a fluid stream, the method comprising:
flowing the fluid stream through a manifold comprising
a conduit comprising an inlet and an outlet and a passage extending therebetween through which the fluid stream to flows, and
one or more sensor ports formed in the conduit between the inlet and the outlet, each of the one or more sensor ports communicating with the passage;
detecting, via one or more sensors equipped to the one or more sensor ports of the manifold, a condition of the fluid stream flowing through the manifold, each of the one or more sensors generating a signal representative of the condition; and
transmitting the signal representative of the condition to a monitoring device.

8. The method of claim 7, wherein the one or more sensor ports each comprise an outwardly extending projection configured to receive a sensor and to place the sensor in contact with the fluid stream flowing through the passage, wherein the manifold comprises one or more baffles positioned within the conduit between the inlet and the outlet, and operable to impede and/or direct the fluid stream flowing through the conduit, and wherein at least one of the one or more sensor ports is positioned downstream of at least one of the one or more baffles.

9. The method of claim 7, wherein the fluid stream comprises a hydraulic fracturing fluid.

10. The method of claim 7, wherein the flow rate of the fluid stream flowing through the manifold is about 100 gal/min to about 10,000 gal/min.

11. The method of claim 7, wherein the flow rate of the fluid stream flowing through the manifold is at least 1000 gal/min.

12. The method of claim 7, further comprising an intervention step comprising introducing one or more additives to the fluid stream in response to the condition detected by the one or more sensors.

13. The method of claim 12, wherein the one or more additives are introduced into a reservoir holding the fluid upstream of the one or more manifolds and/or into the fluid stream downstream of the one or more manifolds, and wherein the one or more additives comprises sand, alcohols, thickening agents, acids, salts, friction reducers, anti-corrosion chemicals, anti-microbials, foaming agents, scale inhibitors, and/or pH buffers.

14. The method of claim 12, wherein the flow of the fluid stream through the manifold is reduced or ceased during the introduction of the one or more additives.

15. The method of claim 7, wherein the condition comprises acidity (pH), oxidation-reduction potential (ORP), temperature, conductivity, total dissolved solids, scaling, corrosion, turbidity, dissolved oxygen, dissolved carbon dioxide, analyte concentrations, pressure, flow rate, specific gravity, and/or oxidizer concentration.

16. The method of claim 7, further comprising repeating the detecting and the transmitting steps at a designated time interval to thereby generate a dataset of fluid stream characteristics.

17. The method of claim 7, wherein the monitoring device comprises:
a control module comprising one or more processing elements,
one or more communication modules, and
a case housing the control module and the one or more communication modules;
wherein the one or more processing elements of the control module receives the signal generated by the one or more sensors and converts the signal to transmittable fluid characteristic data.

18. The method of claim 17, further comprising transmitting the fluid characteristic data to one or more external devices, wherein the control module comprises one or more memory elements that stores the fluid characteristic data for a designated period of time and recalls the fluid characteristic data for transmission after a disconnection event.

19. The method of claim 7, further comprising introducing the fluid stream into a downhole well after the flowing of the fluid stream through the manifold and the detecting the condition of the fluid stream.

20. A computer implemented method for monitoring one or more fluid characteristics of a fluid stream flowing through a manifold, the method comprising:
providing a monitoring device comprising a control module comprising one or more processing elements,
one or more communication modules, and a case housing the control module and the one or more communication modules;
detecting, via one or more sensors equipped to the manifold, a fluid condition of the fluid stream flowing through the manifold;
generating, via the one or more sensors, a signal representative of the fluid condition;
receiving, via the monitoring device, the signal representative of the fluid condition;
converting, via the control module, the signal representative of the fluid condition to transmittable fluid characteristic data;
communicating, via the one or more communication modules, the transmittable fluid characteristic data to one or more external devices.
